(19) United States
(12) Patent Application Publication
(10) Pub. No.: US 2013/0096208 A1
(43) Pub. Date: Apr. 18, 2013

(54) COSMETIC PREPARATION HAVING A CONTENT OF ACYL ARGINATES

(71) Applicants: Manuela Koehler, Hamburg (DE); Bernd Traupe, Kaltenkirchen (DE); Andreas Firyn, Hamburg (DE); Antonia Wagner, Hamburg (DE); Katharina Ropeter, Hamburg (DE)

(72) Inventors: Manuela Koehler, Hamburg (DE); Bernd Traupe, Kaltenkirchen (DE); Andreas Firyn, Hamburg (DE); Antonia Wagner, Hamburg (DE); Katharina Ropeter, Hamburg (DE)

(73) Assignee: Beiersdorf AG, Hamburg (DE)

(21) Appl. No.: 13/578,061

(22) PCT Filed: Feb. 10, 2011
(86) PCT No.: PCT/EP2011/051926

§ 371 Date: Oct. 5, 2012

(30) Foreign Application Priority Data
Feb. 12, 2010 (DE) 10 2010 007 958.8

(51) Int. Cl. A61K 8/43; A61Q 19/00

(52) U.S. Cl. CPC A61K 8/43; A61Q 19/00
USPC 514/784; 514/788; 514/785

(57) ABSTRACT

Cosmetic or dermatological emulsions having a content of one or more acyl arginates of formula (I), where R and R₂ independently of each other can be H and C₅₋₂₂-alkyl, in particular formula (II) or formula (III), wherein said acyl arginates can also be present as salts, for example hydrochlorides.

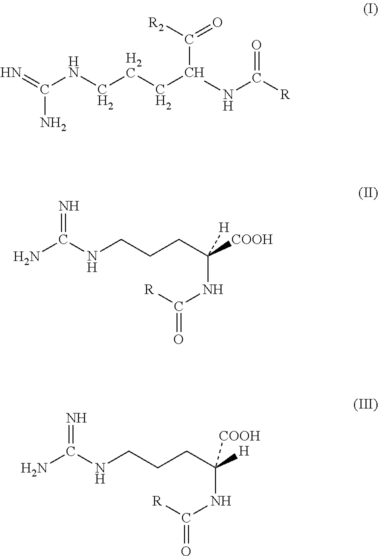

The present invention relates to cosmetic preparations comprising acylarginates.

The heading “cosmetics” can be used to bring together all of the measures which for reasons of esthetics make changes to skin and hair or are employed for body cleansing. Cosmetics therefore signifies the care, enhancement, and beautification of the exterior of the body in order, in a way which can be seen, felt and smelled, to provide pleasure both to those around us and to ourselves. Even millennia ago, cosmetics were used by people for this purpose. The face and lips were colored, valuable oils were used for anointing, and scented water was used for bathing.

Whereas the cleansing action of a shampoo, the improved combability provided by a hair rinse, or the curling of the hair from permanent waving can be established easily and objectively, there are other effects and properties of cosmetic products which are virtually impossible to measure or which can be perceived only on a very individual basis. These include, for example, a particular (invigorating, soft, supple, smooth, etc.) skin sensation, or the softness and suppleness of the skin following application of a cosmetic, and the fullness and bounce of the hair, and so on. In addition, the expectation of consumers is also governed by secondary properties of the product. These include, more particularly, the scent and the color of the cosmetic, and also its packaging, the price, the manufacturer, and the advertising.

Sensory science is the scientific discipline concerned with the evaluation of cosmetic preparations on the basis of sensory impressions. The sensorial assessment of a cosmetic is made on the basis of the visual, olfactory, and tactile impressions.
Visual impressions: all features which can be perceived with the eye (color, shape, structure).
Olfactory impressions: all odorous impressions which can be perceived when air is drawn in through the nose; can frequently be differentiated into initial odor (top note), main odor (middle note, body), and after-odor (finale). Also contributing to the olfactory impression are the volatile compounds released only in the course of application.
Tactile impressions: all sensations of the sense of touch, relating primarily to structure and consistency of the product.

Sensory analysis makes use of the possibility of integrally acquiring the overall sensory impression of a product. Disadvantages of sensory analysis are the subjectivity of the impression, the ease with which test subjects can be influenced, and the associated wide scattering of the results. These weaknesses are nowadays countered by using groups of trained test subjects, screening the testers from one another, and subjecting the analytical data, which in the majority of cases are numerous, to statistical evaluation.

The method of sensory analysis that is most often used in research and development is difference testing. The task in this case is typically confined to the recognition of a specimen that differs from a number of samples or from a control sample. Whereas with different tests only two samples are compared with one another within one test, a ranking test involves ascertaining a sequence of three or more samples, generally according to intensity, quality, popularity or similarity to a comparison sample. This (simple) method is suitable, for example, for preliminary selection of samples in product optimization, and is also used frequently in market research.

The constituents of texture may be described as follows:

Mechanical properties: reaction on stressing, measured kinesthetically

Hardness: Force required to achieve a given deformation

| Foods | Skin care | Fabrics |
|---|---|---|
| Firmness (compression) | Compression force | Compression force |
| Hardness (bite) | Spreading force | Pulling force |

Coherence: Degree to which the sample deforms

| Foods | Skin care | Fabrics |
|---|---|---|
| Coherent | Coherent | Stiffness |
| Tough | Short | Pulling force |
| Brittle (crispy) | | |
| Viscosity | Viscosity | |

Adhesiveness: Force required to remove the sample from a surface

| Foods | Skin care | Fabrics |
|---|---|---|
| Sticky (teeth/gums) | Sticky | Fabric-to-fabric friction |
| Tooth feel | Pulling | Hand friction (pulling) |

Density: Compactness of the cross section

| Foods | Skin care | Fabrics |
|---|---|---|
| Dense/heavy | Dense/heavy | Fullness/soft |
| Airy/fluffy/light | Airy/light | |

Elasticity: Rate of return to initial state after some deformation

| Foods | Skin care | Fabrics |
|---|---|---|
| Elastic, rubbery | Elastic | Elastic |

Geometric properties: perception of the particles (size, shape, orientation) by tactual means

Smoothness: no particles present

Gravelly: small hard particles

Granular: small particles

Chalky/powdery: fine particles (film)

Fibrous: long stringy particles (fiber material)

Lumpy/uneven: large, uniform parts or projections

Moisture properties: perception of water, oil, fat, measured by tactual means, moisture: amount of wetness/oiliness when it is not certain whether it is oil and/or water, moisture release: amount of wetness/oiliness released

| Foods | Skin care | Fabrics |
|---|---|---|
| Juicy | Moisture precipitation | Moisture release |

Oily: amount of liquid fat

Fatty: amount of solid fat

In all cases, the terminology is specific to each type of product, but is based on the underlying rheological properties in accordance with the first texture profile publications (Szczesniak, 1963: Szczesniak et al., 1963; Brandt et al., 1963).

The test persons are selected on the basis of the capacity to differentiate known structural differences in the specific product application for which the test persons are to be trained (solid foods, drinks, semisolid structures, skin care products, fabrics, paper, etc.). As with the majority of other descriptive analysis techniques, the test persons are interviewed in order to determine interest, availability and attitude.

The test persons selected for training are exposed to a wide selection of products in the category under investigation, in order to have a wide reference system. Additionally, the test persons are introduced to the underlying structural principle involved in the structure of the products under investigation. This learning experience provides the test persons with the understanding of the terms of mechanical input forces and resulting stress on the product.

The test persons, accordingly, are able to avoid laborious discussions on redundant expressions, and are able to select the technically most appropriate and most illustrative expressions for evaluating products. The test persons also define all expressions and all procedures for evaluation, and thus partly reduce the variability of the majority of descriptive tests. The reference codes which are used in training the test persons can function later as reference points for approximated scale values, with a further reduction in the variability of the test persons.

The samples are evaluated by each test person independently, using one of the scaling techniques addressed above.

The original texture profile method used an extended 13-point version of the aroma profile scale.

In recent years, however, textile profile test persons have been trained using category scales, linear scales and ME scales, for food texture references for use with a 15-point or 15-cm linear scale. Depending on the nature of the scale used by the test persons, and on the way in which the data have to be treated, the judgments of the test persons may originate from a group consensus, as with the aroma profile method, or from a statistical analysis of the data. For conclusive reports, the data can be shown in table or graph form.

It was an object of the present invention, therefore, to find preparations which, in addition to the criteria customary for cosmetics, such as compatibility, shelf life and the like, also offer cosmetic performances which, while hitherto unrecognized, are essential to the user. In particular, the preparations sought ought to be suitable for use in the body care sector, i.e., in application to the whole body (and hence in delimitation from a pure facial-care application in a relatively large amount), but at the same time are to be sensorily attractive for application in the face area. This is manifested in particular in good spreadability, rapid absorption capacity and a full and caring sensation.

These objects are achieved in accordance with the invention by cosmetic or dermatological emulsions comprising one or more acyl arginates of the formula

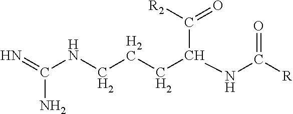

wherein R and R₂, independently of one another, can represent H and C₅₋₂₂-alkyl, in particular

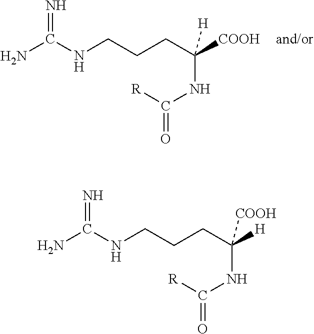

Preference is given to the following compound:

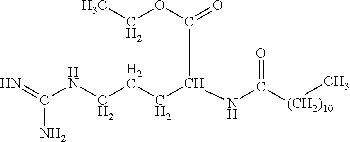

(=lauroyl arginate).

These acyl arginates may also be present as salts, for example, hydrochlorides.

With retention of the features which mark out the present invention, it is possible to obtain cosmetically superior emulsions which are notable for extremely pleasant sensorial properties and cosmetic sensation.

A suitable measurement method for characterizing different formulations in terms of light feeling on the skin, ease of spreading, and nonfatty sensorial properties is that of contact angle measurement (described in Cosmetics & Toiletries, January 2007, Vol. 122, No. 1, pages 20-27 “Correlating Water Contact Angles and Moisturization/Sensory Claims”).

Surprisingly it has now emerged that, through the use of acyl arginates such as, for example, lauroyl arginate (LAE), Ethyl lauroyl arginate HCl, CAS 60372-77-2), the contact angle of a photoprotective formulation is smaller than without. From this it can be concluded that the sensorial properties of the formulation are better than without lauroylarginate.

The preparations of the invention may advantageously constitute water-in-oil emulsions, more preferably oil-in-water emulsions.

Preparations of the invention preferably comprise the acyl arginates at from 0.001% to 10% by weight, preferably 0.05% to 5% by weight, with particular preference 0.05% to 1% by weight, based in each case on the total weight of the preparations.

The preparations of the invention constitute products which are entirely satisfactory in every respect. To a person skilled in the art, it could not have been foreseen that emulsions which, through the targeted setting of the pseudoplastic flow behavior (structural viscosity) and of the thixotropy, exhibit the properties according to the invention, would be able, despite a relatively high yield point, to spread on the skin very easily, in particular, to be rapidly absorbed and nevertheless to be very full and care-imparting.

The cosmetic and/or dermatological formulations of the invention may have a standard composition and may be used for treating the skin and/or the hair, in the sense of a dermatological treatment or of a treatment in beautifying cosmetics. Alternatively they may be used in make-up products in decorative cosmetics.

For application, the cosmetic and/or dermatological formulations of the invention are applied, in a manner customary for cosmetics and dermatological products, to the skin and/or the hair, in a sufficient quantity.

The presence of antioxidants is generally advantageous. The amount of antioxidants (one or more compounds) in the preparations is in that case preferably 0.001% to 30% by weight, more preferably 0.05%-20% by weight, in particular 1%-10% by weight, based on the total weight of the preparation.

The emulsions that are used for the purposes of the invention may be based on one or more of the following emulsifiers:

glyceryl stearate citrate, glyceryl stearate (self-emulsifying), stearic acid, stearate salts, polyglyceryl-3-methylglycose distearate, ceteareth-20, PEG-40 stearate, PEG-100 stearate, sucrose polystearate in combination with hydrogenated polyisobutene, sodium stearoylglutamate, cetearyl alcohol in combination with PEG-40 hydrogenated castor oil, sodium cetearyl sulfate, glyceryl stearate, potassium cetyl phosphate, polyglyceryl-3 sorbitan stearate, silicone polyether copolymers such as, for example, PEG-10 dimethicone, PEG/PPG-14/4 dimethicone, glyceryl stearate citrate and/or sodium stearoyl glutamate, ceteareth-n with n=5-50, sorbitan oleate, distearyldimonium chloride, stearyl dimethicone, lecithin, sodium dihydroxycetyl phosphate, PG-10 stearate, lauryl PEG/PPG-18/18 methicone, cetyl PEG/PPG-10/1 dimethicone, polyglyceryl-2 caprate, isostearyl diglyceryl succinate, cetyl phosphate, sorbitan sesquioleate, polyglyceryl-3 methyl glucose sesquistearate, polyglyceryl-3 methyl glucose distearate, potassium cetyl phosphate, polyglyceryl-2 sesquiisostearate, glyceryl isostearate, behentrimonium chloride, cetearyl glucoside, PEG/PPG-18/18 dimethicone, alkoxylated silicone emulsifiers in general, polyglycerol-derivatized silicone emulsifiers in general, polycaprolactones, sucrose polycottonseedate, acetyl trifluoromethylphenyl valylglycine, hydroxyethylpiperazine, ethane sulfonic acid, hydroxypropyl tetrahydropyrantriol, tocopheryl glycoside, guanosine, polyester-5, tri C14-15 alkyl citrates, PEG/PPG-14/7 dimethyl ether, sodium dodecylbenzene sulfonate, Chrysanthenum parthenium, sodium hydroxypropyl starch phosphate, PEG-60 glyceryl isostearate, succinoglycan, polyglyceryl-2 caprate, dodecene, oxothiazolidinecarboxylic acid, eucalyptol, hydrogenated myristyl olive esters, hydrolyzed wheat protein PG-propyl methylsilanediol.

The lipid phase may advantageously be selected from the following group of substances:
mineral oils, mineral waxes
oils, such as triglycerides in general, and particularly those of capric acid or of caprylic acid, and also natural oils such as castor oil, for example;
fats, waxes, and other natural and synthetic fatty substances, preferably esters of fatty acids with alcohols of low C number, as for example with isopropanol, propylene glycol or glycerol, or esters of fatty alcohols with alkanoic acids of low C number of with fatty acids;
alkyl and/or benzoates;
silicone oils such as dimethylpolysiloxanes, diethylpolysiloxanes, diphenylpolysiloxanes, disiloxane dimethicones, and mixed forms thereof.

The oil phase of the emulsions for the purposes of the present invention is advantageously selected from the group of esters of saturated and/or unsaturated, branched and/or unbranched alkane carboxylic acids with a chain length of 3 to 30 C atoms and saturated and/or unsaturated, branched and/or unbranched alcohols with a chain length of 3 to 30 C atoms, from the group of esters of aromatic carboxylic acids and saturated and/or unsaturated, branched and/or unbranched alcohols with a chain length of 3 to 30 C atoms. Ester oils of this kind may in that case be selected advantageously from the group consisting of isopropyl myristate, isopropyl palmitate, isopropyl stearate, isopropyl oleate, n-butyl stearate, n-hexyl laurate, n-decyl oleate, isooctyl sterarate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, octyl palmitate, octyl cocoate, octyl isostearate, octyldodecyl myristate, isopropyl lauroyl sarcosinate, dibutyl adipate, cetearyl isononanoate, isopropyl myristate, stearyl heptanoate and also synthetic, semisynthetic, and natural mixtures of such esters, e.g., jojoba oil. Further oils which can be used advantageously for the purposes of the present invention are as follows: dialkyl ethers and dialkyl carbonates, especially dicaprylyl ether and/or dicaprylyl carbonate, neopentylglycol diheptanoate, propylene glycol dicaprylate/dicaprate, caprylic/capric/diglyceryl succinate, butylene glycol dicaprylate/dicaprate, C12-13 alkyl lactate, di-C12-C13-alkyl tartrate, triisostearin, dipentaerythrityl hexacaprylate/hexacaprate, propylene glycol monoisostearate, tricaprylin, dimethyl isosorbide, caprylic/capric octyldodecanol, isopropyl palmitate, ethylhexyl cocoate, Butyrospermum parkii, hydrogenated coco-glycerides, Simmondsia chinensis oil, tridecyl stearate, tridecyl trimellitate, dipentaerythrityl hexacaprylate/hexacaprate, lanolin alcohol, Echium lycopsis oil, Shorea robusta butter.

The aqueous phase of the preparations of the invention, where present, comprises optionally, advantageously, alcohols, diols or polyols of low C number, and also ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether, and analogous products, and also alcohols of low C number, e.g., ethanol, isopropanol and 1,2-propanediol.

With very particular advantage the preparations of the invention comprise moisturizers, such as glycerol, for example, more particularly in concentrations of around 2%-50% by weight, the preferred embodiments being distinguished by glycerol contents of 25%-35% and very preferably about 30% by weight. It is, however, entirely possible to reach glycerol contents of around 70% by weight without having then to accept cosmetic or physical disadvantages.

Further moisturizers for the purposes of the invention are as follows: urea and derivatives of urea (for example, Hydrovance®), lactic acid, hyaluronic acid ide, AHAs, EHAs, sodium salt of pyroglutamic acid, xylitol, serine, sodium lactate, ectoin, chitosan, and corresponding derivatives of the stated compounds. Collagen, plankton, extract of Imperata cylindra (Moist 24®), acrylic acid homopolymers (Lipidure-HM®), beta-glucans and corresponding derivatives, and natural oils such as apricot oil, for example. All of the stated substances may optionally also be used in encapsulated form.

Propellants for preparations of the invention that can be sprayed from aerosol containers are the customary, known, volatile, liquefied propellants, examples being hydrocarbons (propane, butane, isobutane), which may be used alone or in a mixture with one another. Compressed air may also be used with advantage.

With advantage it is possible for preparations of the invention, moreover, to include substances which absorb UV radiation in the UVB and/or UVA range, the total amount of the filter substances being, for example, 0.1% to 30% by weight, preferably 0.5% to 10% by weight, more particularly 1.0% to 6.0% by weight, based on the total weight of the preparations, in order to provide cosmetic preparations which protect the hair and/or the skin from the entire range of ultraviolet radiation. They may also serve as sunscreens for the hair or the skin.

The cosmetic preparations of the invention may further comprise, advantageously, although not necessarily, fillers and/or siloxane elastomers, which further improve, for example, the sensorial and cosmetic properties of the formulations, and, for example, bring about or intensify a velvety or silky feeling on the skin.

Advantageous fillers for the purposes of the present invention are starch and starch derivatives, pigments which have neither primarily UV filter effect nor coloring effect (such as boron nitride, etc., for example) and/or Aerosils® (CAS No. 7631-86-9) and/or talc. With particular advantage for the purposes of the present invention it is possible to use the following: tapioca starch, distarch phosphate, aluminum and/or sodium starch octenylsuccinate and the like, polymethylsilesquioxanes, emulsifying silicone elastomers, nonemulsifying silicone elastomers, polymethyl methacrylates, polymethyl methacrylate crosspolymer, polyamides, nylon, polyethylene powders, Polydimethylsiloxyethyl Dimethicone, Acrylates/C10-C30 Alkyl Acrylate Crosspolymer, polysaccharides and derivatives thereof, e.g., celluloses and cellulose derivatives, e.g., hydroxypropylmethylcellulose, methylcellulose, carboxymethylcellulose, hydroxyethylcellulose, hyaluronic acid, xanthan gum, polymers from the group of the polyacrylates, preferably a polyacrylate from the group known as Carbopols, as for example Carbopols of types 980, 981, 1382, 2984, 5984, in each case individually or in combination. Further thickeners advantageous in accordance with the invention are those with the INCI designation Acrylates/C10-C30 Alkyl Acrylate Crosspolymer (e.g. Pemulen TR 1, Pemulen TR 2, Carbopol 1328 from NOVEON) and also Aristoflex AVC (INCI: Ammonium Acryloyldimethyltaurate/VP Copolymer) and also a copolymer of Vinylpyrrolidone and Acrylic Acid, PEG-10 Dimethicone/Vinyl Dimethicone Crosspolymer, Nylon-66, Poly C10-30 Alkyl Acrylate, gums, polysaccharides, cellulose derivatives, phyllosilicates, polyacrylates and/or other polymers, polyacrylamides (Seppigel 305), polyvinyl alcohols, PVP, PVP/VA copolymers, polyglycols, gum arabic, carob flour, tragacanth, karaya, guar gum, pectin, gellan gum, carrageenan, agar, algins, chondrus, xanthan gum, derivatized gums such as Hydroxypropyl Guar (Jaguar® HP 8), citin and chitosan, chondroitin sulfate, starch and starch derivatives, montmorillonite, bentonite, hectorite, laponite, magnesium aluminum silicates such as Veegum®. These may be used as they are or in modified form, such as Stearylalkonium Hectorite. Additionally it is also possible with advantage to use silica gels.

The examples which follow are intended to illustrate the present invention, without restricting it.

| | Example | | | |
|---|---|---|---|---|
| | 1 | 2 | 3 | 4 |
| Glyceryl stearate citrate | 2.5 | 3 | | |
| Glyceryl stearate SE | | | 1 | 1 |
| Cetearyl alcohol + PEG-40 | | | 2.5 | 2.5 |
| castor oil + sodium cetearyl | | | | |
| sulfate | | | | |
| Cetearyl alcohol | | | 1 | |
| Stearyl alcohol | 2 | | | 1 |
| Cetyl alcohol | | | | 3 |
| Acrylates/C₁₀₋₃₀ alkyl acrylate | | | 0.1 | 0.1 |
| crosspolymer | | | | |
| Carbomer | | 0.3 | | |
| Xanthan gum | 0.4 | 0.2 | 0.2 | 0.3 |
| C18-38 acid triglyceride | 0.5 | 1 | | 1 |
| C₁₂₋₁₅ alkyl benzoate | | | 4 | 5 |
| Myristyl myristate | 1 | 2 | | 1 |
| Butylene glycol dicaprylate/ | | 3 | 7 | 3 |
| dicaprate | | | | |
| Propylheptyl caprylate | 5 | 5 | | 12 |
| Dicaprylyl ether | | | 5 | 2 |
| Octyldodecanol | | 2 | | |
| Cyclomethicone | 2 | 3 | 1 | |
| Dimethicone | | | 8 | |
| Emulsifying elastomer | | | 1 | |
| Butylene glycol | 5 | | 3 | |
| Propylene glycol | | 1 | | 5 |
| Glycerol | 3 | 3 | | 3 |
| Octane-1,2-diol | 2 | 5 | 2 | |
| 2-Methyl-1,3-propanediol | | | 3 | 1 |
| Bis-ethylhexyloxyphenol | 2 | 3 | 0.5 | 4 |
| methoxyphenyl triazine | | | | |
| Tris(biphenyl)-1,3,5-triazine | 2 | | | |
| Butyl methoxydibenzoyl- | 3 | 2 | | 4 |
| methane | | | | |
| Homosalate | | | 4 | |
| Octyl salicylate | | | 3 | |
| Octocrylene | 5 | 5 | 8 | 8 |
| Ethylhexyl triazine | | 3 | | |
| Ethylhexyl | 0.5 | | 0.5 | |
| methoxycinnamate | | | | |
| Dioctylbutylamidotriazine | | | | 2 |
| Titanium dioxide | 2 | | | 2 |
| PVP/hexadecene copolymer | 0.2 | 0.1 | 0.2 | |
| Vitamin E acetate | 0.2 | 0.2 | 0.3 | 0.1 |
| Na₂H₂EDTA | 0.1 | 0.2 | 0.2 | 0.2 |
| Perfume | 0.2 | 0.3 | 0.4 | 0.25 |
| Lauroyl arginate | 0.25 | 0.1 | 0.4 | 0.5 |
| Phenoxyethanol | 0.7 | 1 | | |
| Benzethonium chloride | | | 0.05 | |
| Dehydracetic acid | | | | 0.5 |
| Ethylhexylglycerol | | | 0.5 | |
| Sodium hydroxide | q.s. | q.s. | q.s. | q.s. |
| Water | ad | ad | ad | ad |
| | 100.00 | 100.00 | 100.00 | 100.00 |
| | Example | | | |
| | 5 | 6 | 7 | 8 |
| Sodium stearoylglutamate | 0.1 | | 0.4 | 0.1 |
| Sucrose polystearate | 0.8 | | 0.6 | 0.5 |
| Cetearyl alcohol | | | | 1 |
| Copolymer of vinyl- | 0.3 | 0.8 | 0.7 | |
| pyrrolidone and acrylic acid | | | | |
| Acrylate/C10-30 alkyl | 0.2 | | | 0.2 |
| acrylate crosspolymer | | | | |
| Carbomer | | | | 0.2 |
| C12-15 Alkyl benzoate | | | | 2 |
| Butylene glycol | | 3 | | |
| dicaprylate/dicaprate | | | | |
| Octyldodecanol | | 3 | | |
| Dicaprylyl carbonate | 3 | | | |
| Caprylic/capric triglyceride | | | | 2 |
| Isodecyl neopentanoate | 2 | | | |
| Cyclomethicone | | 8 | 3 | 24 |
| Dimethicone | | | 2 | |
| Dimethicone crosspolymer | | 1 | | 3 |
| Glycerol | 5 | 5 | 7 | 1 |
| Polymethylsilsesquioxane | | | 5 | |
| Hexane-1,2-diol | 2 | 3 | | |
| Bis-ethylhexyloxyphenol | 1 | | 2 | 2.5 |
| methoxyphenyl triazine | | | | |
| Ethylhexyl triazine | | | | 1 |
| Butyl methoxydibenzoyl- | 3 | 2 | | |
| methane | | | | |
| Diethylamino hydroxy- | | 2 | 5 | 3 |
| benzoyl hexyl benzoate | | | | |
| Polysilicone-15 | 2 | | | |
| Ethylhexyl methoxy- | | | 5 | |
| cinnamate | | | | |
| Octyl salicylate | | 5 | | 3 |
| Homosalate | | | | 5 |
| Octocrylene | 4 | 8 | 2 | 5 |
| Phenylbenzimidazole sulfonic | 2 | | | |
| acid | | | | |
| Ethanol | | 4 | | 3 |
| Lauroyl arginate | 0.3 | 0.1 | 0.25 | 0.4 |
| Methylisothiazolinone | | | 0.05 | |
| DMDM Hydantoin | 1 | | | 0.5 |
| 1,2-Hexanediol | | 0.8 | | |
| Pentylene glycol | | 3 | | |
| Water | ad | ad | ad | ad |
| | 100.00 | 100.00 | 100.00 | 100.00 |

| | Hydrodispersion | |
|---|---|---|
| | 9 | 10 |
| Sodium carbomer | 0.30 | |
| Copolymer of vinylpyrrolidone and acrylic | | 1.0 |
| acid | | |
| Sodium stearoyl glutamate | | 0.1 |
| Acrylates/C10-30 alkyl acrylate crosspolymer | 0.10 | |
| Xanthan gum | | 0.50 |
| Dimethicone/vinyl dimethicone crosspolymer | | |
| Hexyl 2-(4′-diethylamino-2′- | 2.00 | 0.25 |
| hydroxybenzoyl)benzoate | | |
| Terephthalidene dicamphorsulfonic acid | | 2.0 |
| Ethylhexyl triazine | | 4.00 |
| Butyl methoxydibenzoylmethane | | 4.2 |
| Titanium dioxide | | 0.50 |
| 1,2-Hexanediol | | 0.50 |
| Methylpropanediol | 3 | 0.5 |
| C₁₂₋₁₅ Alkyl benzoate | | 2.00 |
| Oleic acid | | 0.5 |
| Butylene glycol dicaprylate/dicaprate | 6.00 | 4.00 |
| PVP hexadecene copolymer | 0.50 | 0.50 |
| Glycerine | 5.00 | 10.00 |
| Butylene glycol | | 20 |
| 1,2-Octanediol | 0.1 | |
| Vitamin E acetate | 0.5 | 0.50 |
| Panthenol | 0.25 | 1.50 |
| Trisodium EDTA | 0.20 | |
| Ethanol | | 3.00 |
| Lauroylarginate | 0.5 | 0.2 |
| Methylisothiazolinone | | 0.09 |
| DMDM Hydantoin | 0.5 | |
| Perfume, dyes, | q.s. | q.s. |
| Water | ad 100 | ad 100 |

1.-6. (canceled)

7. A cosmetic or dermatological emulsion, wherein the emulsion comprises one or more acyl arginates of formula 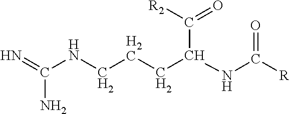
wherein R and R₂, independently of one another, represent H or C₅₋₂₂-alkyl.

8. The emulsion of claim 7, wherein the emulsion comprises at least one of

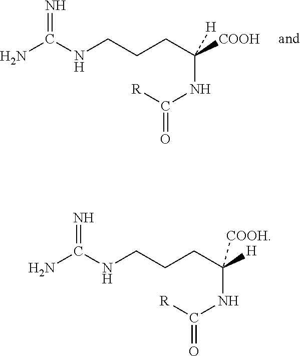

9. The emulsion of claim 7, wherein the one or more acyl arginates are present as salts.

10. The emulsion of claim 9, wherein the one or more acyl arginates are present as hydrochlorides.

11. The emulsion of claim 7, wherein the one or more acyl arginates comprise a compound of formula

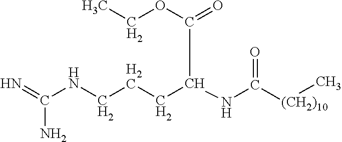

12. The emulsion of claim 7, wherein the emulsion comprises from 0.001% to 10% by weight of the one or more acyl arginates, based on a total weight of the emulsion.

13. The emulsion of claim 12, wherein the emulsion comprises from 0.05% to 5% by weight of the one or more acyl arginates.

14. The emulsion of claim 13, wherein the emulsion comprises not more than 1% by weight of the one or more acyl arginates.

15. The emulsion of claim 7, wherein the emulsion is an oil-in-water emulsion.

16. The emulsion of claim 7, wherein the emulsion is a water-in-oil emulsion.

17. The emulsion of claim 7, wherein the emulsion further comprises glycerol.

18. The emulsion of claim 17, wherein the emulsion comprises from about 2% to 50% by weight of glycerol, based on a total weight of the emulsion.

19. The emulsion of claim 18, wherein the emulsion comprises from 25% to 35% by weight of glycerol.

20. The emulsion of claim 18, wherein the emulsion comprises about 30% by weight of glycerol.

21. A cosmetic or dermatological emulsion, wherein the emulsion comprises from 0.05% to 1% by weight one or more acyl arginates of formula 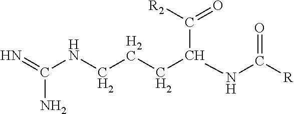
wherein R and R₂, independently of one another, represent H or C₅₋₂₂-alkyl and from 25% to 35% by weight of glycerol, each based on a total weight of preparation.

22. The emulsion of claim 21, wherein the emulsion comprises at least one of

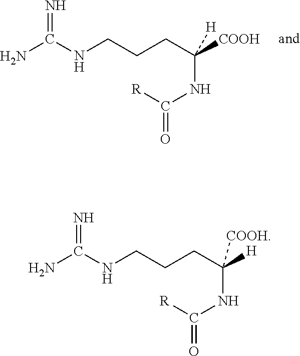

23. The emulsion of claim 21, wherein the one or more acyl arginates comprise a compound of formula

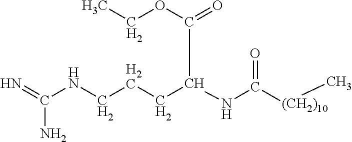

24. The emulsion of claim 23, wherein the emulsion is an oil-in-water emulsion.

25. The emulsion of claim 23, wherein the emulsion is a water-in-oil emulsion.

26. The emulsion of claim 23, wherein the emulsion comprises about 30% by weight of glycerol.
